(19) United States
(12) Patent Application Publication
(10) Pub. No.: US 2020/0300836 A1
(43) Pub. Date: Sep. 24, 2020

(54) SYSTEMS AND METHODS FOR POINT-OF-CARE DETECTION OF POTASSIUM

(71) Applicant: POLYMER TECHNOLOGY SYSTEMS, INC., Whitestown, IN (US)

(72) Inventors: Christopher Dailey, Whitestown, IN (US); Gary L. Hughes, Camby, IN (US); Keith Moskowitz, Westfield, IN (US)

(73) Assignee: POLYMER TECHNOLOGY SYSTEMS, INC., Whitestown, IN (US)

(21) Appl. No.: 16/601,433

(22) Filed: Oct. 14, 2019

Related U.S. Application Data
(60) Provisional application No. 62/744,685, filed on Oct. 12, 2018.

(51) Int. Cl. G01N 33/487; G01N 21/64; G01N 33/49; G01N 27/327; G01N 27/30

(52) U.S. Cl. CPC G01N 33/48707; G01N 21/6486; G01N 2021/7759; G01N 27/3272; G01N 27/301; G01N 33/49

(57) ABSTRACT

Systems and methods for point-of-care determination of blood potassium are provided. The approaches are based on the reaction of ADP and PEP in the presence of potassium ion and pyruvate kinase to produce pyruvate and ATP. In one method, the produced pyruvate is reacted with phosphate and mediator, in the presence of pyruvate oxidase, to yield acetylphosphate and reduced mediator, and the reduced mediator is used to determine the blood potassium using conventional electrochemical methods. In an alternative method, the produced pyruvate is reacted with phosphate and oxygen in the presence of pyruvate oxidase to yield acetylphosphate and hydrogen peroxide, and the hydrogen peroxide is measured optically in accordance with known methods.

### PRIORITY

This application claims the benefit of U.S. provisional application No. 62/744,685 filed Oct. 12, 2018, which is hereby incorporated by reference. Applicant hereby incorporates herein by reference the US and EP patents cited herein.

### BACKGROUND

Testing potassium levels in the blood is typically something that is not done outside a clinical lab. Test methods include using ion selective electrodes or an enzymatic method (pyruvate kinase). The instrumentation for such testing is expensive and not suitable for home use or self-testing.

U.S. Pat. No. 7,410,755 provides for determining ADP in an enzyme-coupled reaction. In this method, pyruvate kinase and phosphoenolpyruvate are combined in the assay mixture and react with ADP to form ATP and pyruvate. Pyruvate oxidase and its cofactors FAD and TPP are used to transform the pyruvate to acetyl phosphate and hydrogen peroxide. The hydrogen peroxide is then detected by catalyzing its reaction with a fluorescent dye using horseradish peroxidase. See, U.S. Pat. No. 7,410,755, col. 1, lines 43-51. See also, EP 0 274 425.

U.S. Pat. No. 4,705,749 describes an alternative method for determining ADP. The first step also comprises reacting ADP with phosphoenolpyruvate (PEP) in the presence of pyruvate kinase (PK). PK dephosphorylates the PEP to form pyruvate and ATP. In a second step, the pyruvate is reacted with NADH and H+ in the presence of lactate dehydrogenase (LDH). LDH converts the pyruvate to lactate, while NADH is oxidized to NAD+. The decrease in NADH is monitored directly in ultra-violet light. See, U.S. Pat. No. 4,705,749, col. 3, lines 25-36.

Systems and methods are known for performing a potassium enzymatic assay on a test sample fluid also based on an ADP reaction approach. In this reaction scheme, the rate of change from NADH to NAD+ is proportional to the amount of potassium in the sample. However, because the wavelength to detect the disappearance of NADH is too low (340 nm) for reflectance technology, this system is unsuitable for a portable, point-of-care (POC) analyzer.

### SUMMARY

In one embodiment, a test strip for detecting potassium in a blood sample includes a working electrode and a reference electrode. Additionally, the test strip includes a testing area, including the working electrode and the reference electrode. Additionally, the test strip includes a reagent mixture, the reagent mixture deposited in association with one of the working electrode, the reference electrode, and the testing area, the reagent mixture including Adenosine diphosphate (ADP), Phosphoenolpyruvate, Pyruvate Kinase, Mg2+, Phosphate, a Mediator, and pyruvate Oxidase. In one alternative, the mediator is nitrosoaniline. In another alternative, the reagent further includes lithium. Alternatively, the Pyruvate Kinase is derived from Bacillus stearothermophilus. In another alternative, the test strip detects potassium according to the equation:

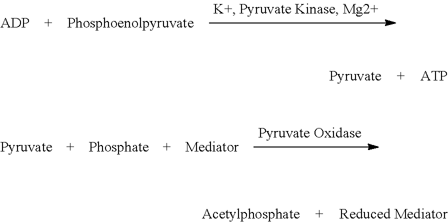

where the reduced mediator represents a charge detectable by a meter.

In another alternative, the mediator is selected from the group consisting of 4-nitrosoaniline, lithium ferricyanide, sodium ferricyanide, and rubidium ferricyanide. Alternatively, the pH of the reagent mixture is 6.5 and is achieved by adding LiOH. In another alternative, the reagent mixture further includes polyethelyene oxide and Triton X-100. Alternatively, the working electrode and the reference electrode are interdigitated.

In one embodiment, a method for electrochemically detecting potassium in a blood sample includes reacting ADP with phosphoenolpyruvate in the presence of potassium ion and pyruvate kinase to produce pyruvate and ATP. The method further includes reacting the produced pyruvate with phosphate and mediator in the presence of pyruvate oxidase to yield acetylphosphate and reduced mediator. The method further includes, electrochemically measuring the reduced mediator. The method further includes correlating the amount of reduced mediator to an amount of potassium. In another alternative, the mediator is potassium ferricyanide and the reduced mediator is potassium ferrocyanide. Alternatively, the mediator is 4-nitrosoaniline. In another alternative, the mediator is lithium ferricyanide.

In one embodiment, a method of detecting potassium includes reacting ADP with phosphoenolpyruvate in the presence of potassium ion and pyruvate kinase to produce pyruvate and ATP. The method further includes reacting the produced pyruvate with phosphate and oxygen in the presence of pyruvate oxidase to yield acetylphosphate and hydrogen in positive ion form, whether bound or unbound. The method further includes measuring the hydrogen. The method further includes correlating the amount of hydrogen to an amount of potassium. In one alternative, the hydrogen is hydrogen peroxide. In another alternative, the hydrogen is bound with a mediator. Alternatively, the optically measuring comprises reacting the hydrogen peroxide with a Trinder Reagent. In another alternative, the method includes optically measuring and the optically measuring comprises measuring fluorescence. Alternatively, the optically measuring comprises reacting the hydrogen peroxide with a Trinder Reagent. In another alternative, the reacting steps are performed using a whole blood sample. Alternatively, the reacting steps are performed using a serum sample. In another alternative, the reacting steps are performed using a plasma sample. Alternatively, the reacting steps are performed at the point of care. Optionally, the reacting steps are performed using a point-of-care device. Alternatively, the reacting steps are performed using a whole blood sample. Optionally, the reacting steps are performed using a serum sample. Alternatively, the reacting steps are performed using a plasma sample.

In one embodiment, a test strip for detecting potassium in a blood sample includes a working electrode and a reference electrode. The test strip includes a testing area, including the working electrode and the reference electrode. The test strip includes a reagent mixture, the reagent mixture deposited in association with one of the working electrode, the reference electrode, and the testing area, the reagent mixture including Adenosine diphosphate (ADP), Phosphoenolpyruvate, Pyruvate Kinase, a Mediator, and pyruvate Oxidase. In one alternative, the reagent mixture includes Mg2+ and Phosphate. In another alternative, the mediator is nitrosoaniline. Alternatively, the reagent further includes lithium.

### BRIEF DESCRIPTION OF THE DRAWINGS

FIG. 1 shows the electrochemical tests according to one embodiment of a disclosed method provides a highly linear response for mM pyruvate;

FIG. 2 shows one embodiment of an electrochemical test strip for determining potassium;

FIG. 3 shows a table of the concentrations for interference testing;

FIGS. 4 and 5 show results from interference testing;

FIG. 6 shows a table of t-test considerations for the interferents;

FIG. 7 shows the results of testing twenty replicates of potassium solutions in serum;

FIG. 8 shows a graphical representation of the % CV for low, medium and high potassium concentrations;

FIG. 9 shows this bias plot of the accuracy compared to results on a Cobas Integra 400 (which is a standard testing device in the industry);

FIG. 10 provides the graph of the method comparison for the electrochemical strips v. the Cobas Integra;

FIG. 11 displays both the calibration and verification samples on the same chart;

FIG. 12 shows the results of testing twenty replicates of potassium solutions in a buffered solution;

FIG. 13 shows a graphical representation of the % CV for each of the samples;

FIG. 14 shows a box and whisker plot of the potassium detection data; and

FIG. 15 shows a bias plot of potassium response.

### DESCRIPTION

Certain terminology is used herein for convenience only and is not to be taken as a limitation on the embodiments of the systems and methods for the detection of point of care potassium. Disclosed are systems and methods for detecting blood potassium that may be used by a POC device. A particular advantage of the methods is that they may be performed at the point of care, using a point-of-care device of the types known in the art for electrochemical determinations. The methods also may be performed using blood, as whole blood, serum or plasma, any of which again may be accomplished in a point-of-care device. The potassium results may be obtained quickly and accurately from a portable, low cost, POC analyzer. The testing requires only a fingerstick of blood, rather than a venous draw, and the testing may therefore be performed as self-testing at home. Disadvantages of other potassium methods, including expensive, non-portable instrumentation, are avoided.

Previously it has been that it is possible to detect pyruvate either electrochemically or optically. Provided herein is an approach which leverages the detection of pyruvate as a means to determine blood potassium. Because potassium is required for pyruvate kinase activity, and pyruvate kinase produces pyruvate, the present approaches useful in detecting potassium. The reaction schemes of the present disclosure allow for detecting potassium either electrochemistry or optically.

The detection of potassium electrochemically starts with the generation of pyruvate from ADP:

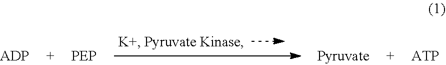

The pyruvate is then reacted with phosphate and mediator, in the presence of pyruvate oxidase, to yield acetylphosphate (ACP) and reduced mediator:

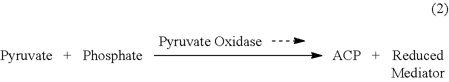

In some embodiments, a suitable mediator would be one, such as ferricyanide (which is reduced to ferrocyanide), known to be useful in electrochemical measurement systems. In other embodiments, a preferred mediator is cesium ferricyanide or a nitrosoaniline derivative. It has been shown herein that nitrosoaniline may function the best in many scenarios, but this should not thought to be at the exclusion of the other mediators identified. Various other mediators known in the art for use in electrochemical methods, are also useful in the present methods. The reduced mediator is used to determine the blood potassium using conventional electrochemical methods.

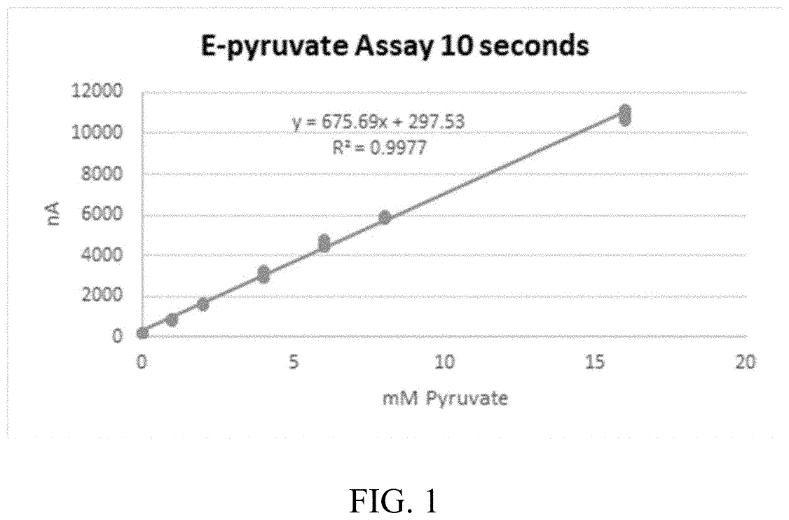

As shown in FIG. 1, the electrochemical tests according to this method provide a highly linear response for mM pyruvate.

The detection of potassium optically also starts with the production of pyruvate from ADP in accordance with Equation (1). The produced pyruvate is then reacted with phosphate and oxygen in the presence of pyruvate oxidase to yield acetylphosphate and hydrogen peroxide:

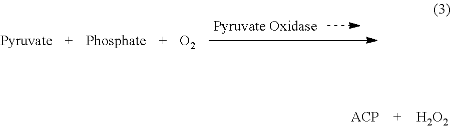

The hydrogen peroxide is then measured optically in accordance with well-known methods, e.g.:

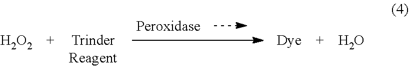

The electrochemical and optical reaction schemes disclosed herein are both suitable for use in conventional fashion with calibration curves. As FIG. 1 demonstrates, a calibration curve may be developed which correlates the response signal to a concentration of potassium. A series of tests are conducted at varying concentrations of potassium in order to establish a correlation to signal response for the desired range of concentrations. The results shown in FIG. 1, for example, provide a calibration curve for the electrochemical test correlating the electrochemical signal in nanoamps (nA) with the concentration of potassium in millimolar (mM) units. Similar calibration curves may be derived for optical tests, e.g., fluorescence or reflectance, in accordance with procedures known in the art. In the alternative, an algorithm, look-up table, etc. may be derived which similarly correlates the response signal to the concentration of potassium.

Required reagents are provided in a form suitable for combination with the ADP. In addition to the identified reactants, other components may be present in the reagent system(s), including for example co-factors, binders, preservatives, diluents, and such other excipients as known in the art to be useful. Examples of such other excipients are described in the US and EP patents incorporate herein. The reagents are provided either as a single system, or as two or more systems, and may be included on test strips, in sample shakers, or in other appropriate carriers, and test vehicles. The ADP is incubated with the reagents and appropriate measurements are taken in accordance with known electrochemical or optical methods.

In many embodiments, a mediator used is Nitrosoaniline derived. A ferricyanide whose counter ion is not potassium or sodium will work well as a mediator. In some embodiments, a two-mediator system using nitrosoanaline and 1-methoxy PMS. 1-methoxy PES is a potential mediator as well. In many scenarios having a mediator that does not inhibit enzyme activity of the pyruvate kinase is preferable. In some alternatives, lithium is added to the system and a pyruvate kinase from Bacillus stearothermophilus is used, which is significantly more selective for potassium. This reduces sodium interference.

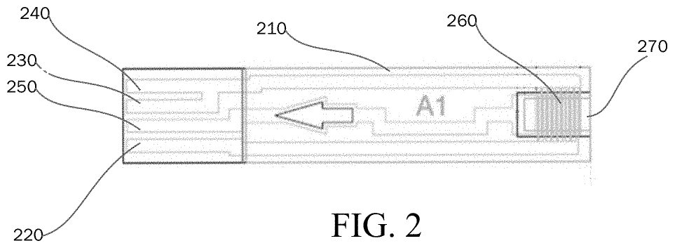

FIG. 2 shows one embodiment of an electrochemical test strip for determining potassium. Strip 210 is configured to be inserted into a meter. Leads 220-250 provide for interface with electrical systems of the meter that apply current, voltage, and detect resistance or other electrical activities. Lead 220 provides for attachment to the counter electrode. Leads 230, 240 provide for a working electrode, with the combination of lead 230 and lead 240 providing for a strip insertion detection. Interdigitated electrodes 260 are in sample area 270 and have one of the embodiments of the reagents discussed herein either on some combination of electrodes, in the sample entrance, or in the sample area. This type of strip may be modified with many of the reagents discussed herein in order to create a suitable test for the electrochemical detection of potassium. In many embodiments discussed herein, this strip and modifications thereof provide for a strip that tests for potassium.

In many embodiments, there may be some interference from certain parts of the blood. For some embodiments, a reaction scheme is presented below. Since this reaction scheme has been shown to be feasible, probable interferences must be reduced or eliminated.

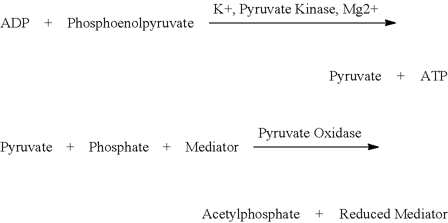

When testing for ions such as potassium, it must be ensured that there is not interference from other, similar ions. For potassium assays, sodium and ammonium are possible major interferents. As part of developing this assay, an interference study examined the effects of the presence of sodium and ammonia in the serum sample at worst-case, pathological values for the potassium assay, assessing the impact these interferences have on the risk to the product development.

As a side note, ammonia and ammonium are often used interchangeably in diagnostic literature, although in reality they are two different compounds in equilibrium in solution. The relative concentrations are dependent on the pH of the solution. Diagnostic assays for “ammonia” actually measure the ammonium ion. In the potassium assay, the ammonium ion is the interferent in serum.

In one example of a test for determining how to reduce interference of sodium and ammonia, a test was established. In short, an interferent is spiked into a sample and then tested against a control sample. There could be a positive interference if the interferent caused an increase in analyte concentration, or a negative interference where the interferent caused a decrease in concentration. For the potassium assay, both sodium and ammonia will show positive interferences if an interference exists.

As part of the testing, first the sensor was prepared. A new lot of potassium sensors were made and calibrated with serum samples. Reagents were hand deposited on the sensors. Generally, sensors such as those shown in FIG. 2 were used and could be used. Such sensors, have interdigitated electrodes and the reagents may be deposited on such electrodes.

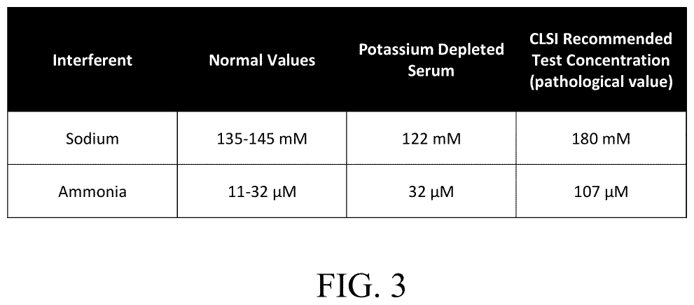

As part of the testing, sodium and ammonia concentrated spikes were prepared. Spiking solutions are prepared at 20× the desired concentration so that no more than 5% can be spiked into the sample. For example, 1 M sodium spiking solution was prepared by adding 0.2922 g of sodium chloride to 5 mL of potassium depleted serum. Potassium depleted serum is from ProMedDx (affiliate of Precision for Medicine) Scan #2748322. For example, 1.6 mM ammonium spiking solution was prepared by adding 0.0004 g ammonium chloride to 5 mL of potassium depleted serum. FIG. 3 shows a table of the concentrations for interference testing.

Subsequently, serum sample (or modified blood sample) was prepared. For some embodiments of the testing, the phosphoenolpyruvate (PEP) has been removed from the reagent and added to the buffer or serum in order to prevent the reaction from occurring prior to application of the sample. This is performed due to the presence of trace amounts of potassium and sodium in the reagents. Even though they are highly pure, a small amount can cause erroneous results. Because of this problem, one ingredient has been isolated, here the PEP, to keep the reaction from occurring during the manufacturing of the strip. By removing the reactant from the reagent, the reaction cannot occur during strip manufacturing. The final potassium assay has the entire reagent dried down on the strip but eliminating all trace amounts of potassium and sodium. There are methods to remove potassium and sodium from reagents that can be added to the system production that are known to those of ordinary skill in the art. Interference testing usually entails testing a low and high-level analyte with the interferent. Serum samples targeting 3.5 and 7 mM potassium are prepared.

In one example, the experimental setup involved: 1) For each level of potassium prepare three (3) 1 mL aliquots. There will be an aliquot for the control, for sodium interference and ammonium interference. 2) Add 50 μL of blank serum to the control aliquot. 3) Add 50 μL of sodium spike and ammonium spike to the respective samples. 4) Mix thoroughly before testing.

In one example, the interference testing involved the following steps: 1) Set up the custom potentiostat test stand. 2) Test each serum sample 20 times for N=20. 3) Analyze each serum solution for potassium, sodium and ammonia on the reference analyzer (Cobas Integra 400).

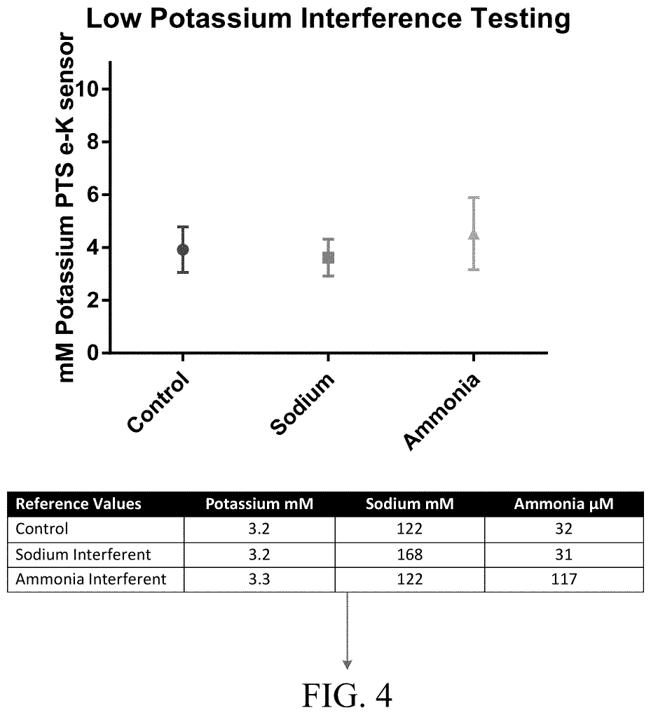

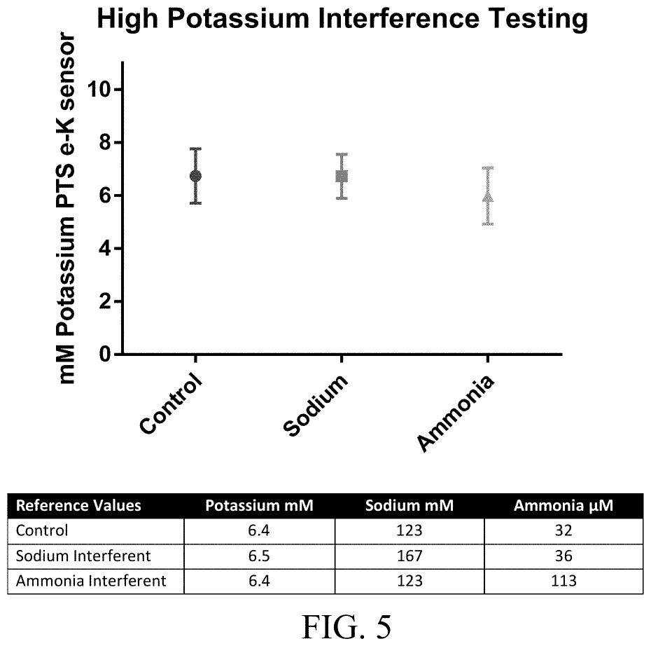

The results are provided in FIGS. 4 and 5. The prepared samples were tested on the potassium electrochemical strips to investigate the extent of any interference. Control and interferent samples were also measured on the Cobas Integra 400 reference analyzer to determine actual concentrations. The graphs below indicate there is no statistical difference with the addition of sodium and ammonia in serum at pathological concentrations.

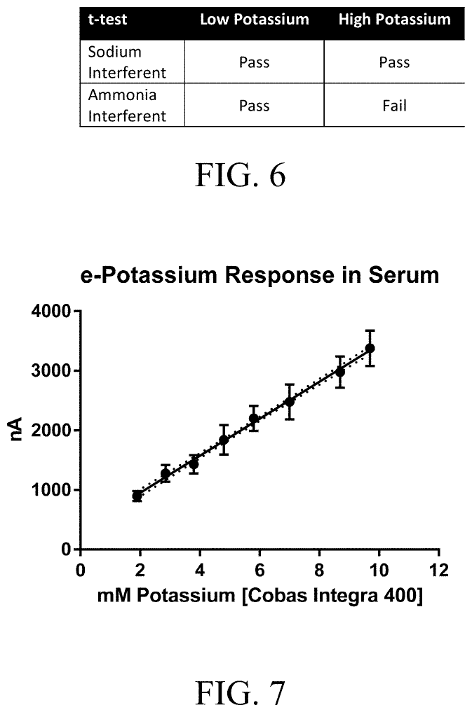

To evaluate interference in this potassium assay, the statistical tool of the t-test was used. The t-test evaluates two populations of data and determines with 95% confidence whether the means of the two populations are statistically different. The table below shows that in all but one instance, the means of the interferent data and the control are the same. When testing the ammonia spiked into the high potassium sample, the t-test concluded that the mean was statistically different than the control (95% confidence). However, it must be observed that this sample mean was lower than the control. As stated previously, both ammonia and sodium will be positive interferences if they interfere. Therefore, it is understood that ammonia is not an interferent in this testing. It is most likely that the imprecision observed for this sample gave a lower mean and thus failed the t-test. FIG. 6 shows a table of t-test considerations for the interferents.

Both ammonium and sodium ions have the capacity to be a cofactor for pyruvate kinase in place of potassium. In serum, sodium concentrations are usually around 140 mM while potassium concentrations are much lower around 4 mM. To keep the sodium from interfering, we have selected a pyruvate kinase from Bacillus stearothermophilus, which is significantly more selective for potassium. In addition, some literature indicates that the addition of lithium ions act as a competitive inhibitor to sodium's interaction with pyruvate kinase; but lithium is not a cofactor. Lithium phosphate is used as a buffer in the potassium assay to further eliminate sodium interference.

Ammonium interference is more difficult to eliminate. Some literature claims that ammonium reacts with pyruvate kinase on an equimolar basis as potassium. However, ammonium is in very low concentrations in the serum. Normal concentrations are between 11-32 μM. If it is true that ammonium reacts with pyruvate kinase on an equimolar basis, then a high “normal” sample would add a negligible 0.032 mM to the potassium result. Even at pathological values, the theoretical interference would only be 0.1 mM positive bias.

An added advantage that the disclosed electrochemical assay has against interference from ions is the speed of the assay. A more selective pyruvate kinase and a faster reaction rate (<70 seconds) does not allow the sodium, and possibly ammonium, to have a chance to interact in a meaningful way before the test is finished. At this juncture, we do not observe interferences from either sodium or ammonium.

Therefore, based on this proof of concept, a strip-sensor for testing for potassium has been established with reduced or eliminated interference from sodium and ammonium. As disclosed, the addition of lithium and the usage of pyruvate kinase from Bacillus stearothermophilus results in improved performance by the reduction of interference from sodium and as observed, ammonia has a minimal impact, if any, on the performance of the strip-sensor.

As previously stated, one embodiment the reaction scheme for electrochemical potassium is presented below.

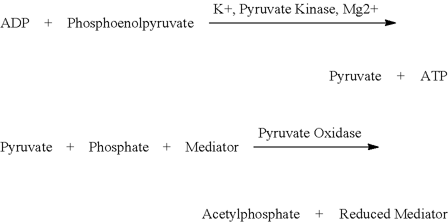

In this assay system, the amount of potassium determines how much pyruvate is generated in a given amount of time (step 1). The pyruvate produced can then be measured electrochemically using pyruvate oxidase (step 2). Since potassium is a cofactor of interest, all substrates should be in excess and in the proper ratios for maximum pyruvate kinase reactivity making potassium the limiting factor.

In order to bring, the chemistry to an electrochemical format, mediators needed to be screened which would react with pyruvate oxidase to provide a detectable signal. In total, eighteen different mediators were screened. If the mediator was compatible with pyruvate oxidase, further experiments were conducted to evaluate the pyruvate kinase reaction. Surprisingly, many of the mediators inhibited the pyruvate kinase. This was not expected because these mediators are used in many other diagnostic assays. However, nitrosoaniline was determined to be both reactive with the pyruvate oxidase and compatible with pyruvate kinase.

In one experiment, a strip-sensor is prepared using a compound potassium reagent. Using a pipettor, 4 μL of reagent onto each sensor. As previously stated, the sensors were similar to that shown in FIG. 2. The sensors included gold interdigitated electrodes. Subsequently, the sensors were dried at 50° C. in convection oven for 5 minutes.

In this testing, the phosphoenolpyruvate (PEP) has been removed from the reagent and added to the buffer or serum in order to prevent the reaction from occurring prior to application of the sample. This is required due to the presence of trace amounts of potassium and sodium in the reagents. Even though they are highly pure, a small amount can cause erroneous results. Of course, when used on an actual sample, produced from a human and immediately applied, this would not be needed. Because of this problem, one ingredient was isolated, here the PEP, to keep the reaction from occurring during the manufacturing of the strip. By removing the reactant from the reagent, the reaction cannot occur during strip manufacturing. This testing is to prove the concept that the test works. There are methods, known to those of ordinary skill in the art, to remove potassium and sodium from reagents that can be implemented for a commercial test strip.

The next step in the testing was to thaw frozen potassium depleted serum in water bath at 37° C. until serum is liquid. Potassium depleted serum is from ProMedDx (affiliate of Precision for Medicine) Scan #2748322.

The next step in the testing was to prepare a stock of 10 mM potassium serum by adding potassium chloride to the potassium depleted serum. Prepare a series targeting 2, 3, 4, 5, 6, 7.3, 9, and 10 mM potassium at 2 mL each. For curve verification, prepare another series of potassium in serum at 1.5, 2.5, 3.5, 4.5, 5.5, 6.5, 7.5, 8.5 and 10 mM potassium was prepared. To each of the serum solutions, 30 mM PEP tricyclohexylammonium salt (0.0297 g in 2 mL) was added. Subsequently, each serum solution was verified across 14 sensors (N=14) and the data was evaluated.

Because potassium is a cofactor of pyruvate kinase, the amount of signal is based on the amount of pyruvate generated over a given amount of time. FIG. 7 shows the results of testing twenty replicates of potassium solutions in serum. This linear regression was used as a calibration for these sensors. Error bars are representative of standard deviation.

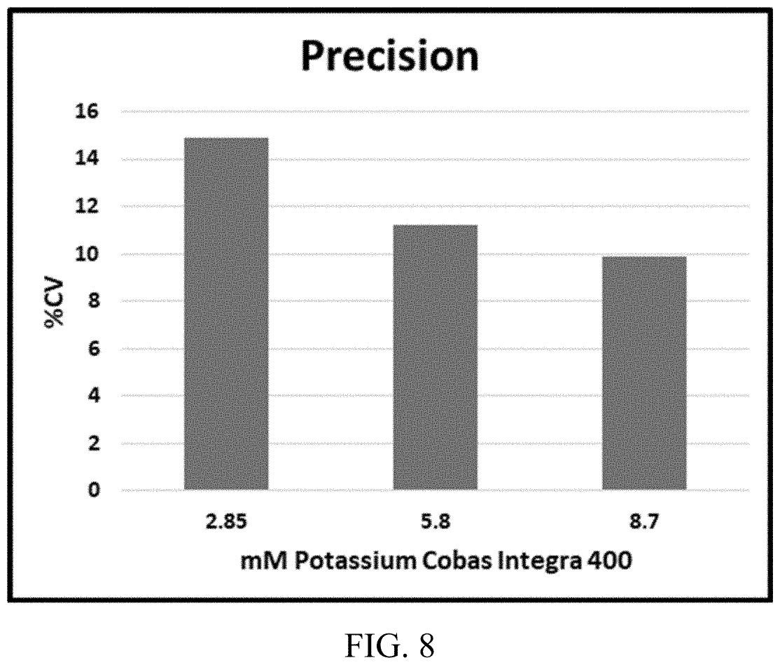

By using the equation of the linear regression, the observed potassium values were able to be calculated. From these values the precision was computed for low, medium, and high potassium concentrations. The overall precision was 12.0% CV. Some of the imprecision is due to flyers from hand deposited sensors. FIG. 8 shows a graphical representation of the % CV for low, medium and high potassium concentrations.

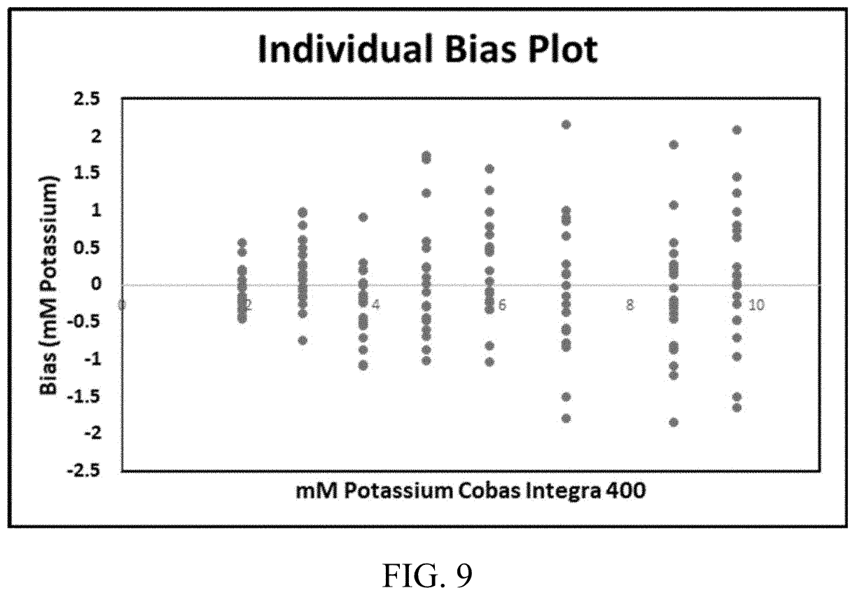

By examining the linear regression line, it can be observed that the bias is well centered around zero. This illustrates the accuracy of the assay. FIG. 9 shows this bias plot of the accuracy compared to results on a Cobas Integra 400 (which is a standard testing device in the industry).

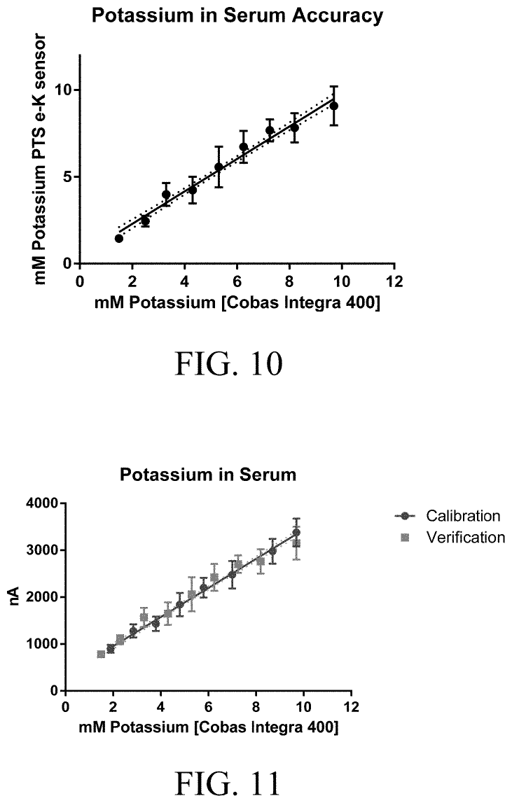

Using the initial calibration curve, another set of serum samples was used to further examine the accuracy of the assay. Serum was spiked at levels 1.5, 2.5, 3.5, 4.5, 5.5, 6.5, 7.5, 8.5 and 10 mM potassium. FIG. 10 provides the graph of the method comparison, while FIG. 11 displays both the calibration and verification samples on the same chart. For FIG. 10, the curve verification with 9 different serum samples has a slope of y=0.9274+0.4812.

The precision shown meets the overall goals for feasibility and will be improved as greater precision is brought to the production of the sensor-strips. In this example, the timing for the testing was 20 seconds and will still be optimized along with the reagent. The timing is critical as if the reaction was allowed to go on indefinitely, eventually the same amount of signal would be generated regardless of the potassium concentration. The amount of potassium determines the rate of pyruvate generated, not the absolute amount. Because of this, the timing of the assay needs to be tuned such that there is sufficient differentiation between the smallest and largest concentration of potassium. The timing of the testing will change, and the setup and reagents are optimized.

The accuracy of this electrochemical potassium assay is quite good. The bias is well centered around zero. In addition, additional serum samples verify the calibration curve.

In many embodiments, the strategy for electrochemical testing was to optimize the PEP, ADP and magnesium which interact with pyruvate kinase, while keeping potassium in excess. When the substrates and cofactors have been optimized, the potassium can be removed from the system and demonstrate a dose response. Optimization was conducted with potassium ferricyanide as the mediator. As identified, potassium ferricyanide may not be the optimal mediator, however, these tests show that the system performs well, and the principles established, provide for the clear substitution of the preferred mediators identified here.

Multiple mediators were screened for the pyruvate reaction. If the mediator was compatible with pyruvate oxidase, further experiments were conducted to examine the pyruvate kinase reaction. Surprisingly, many of the mediators inhibited the pyruvate kinase. This was not expected because these mediators have been used in many other diagnostic assays. The unforeseen interference with pyruvate kinase took up much time in troubleshooting. However, once it was understood that some mediators inhibited the pyruvate kinase, a secondary screening was set up to eliminate those that would not work. In many embodiments, Nitrosoaniline was chosen because it does not interfere with pyruvate kinase and reacts with pyruvate oxidase. The following illustrates the work with the nitrosoaniline mediator and demonstrates that potassium can be detected on an electrochemical sensor.

For many embodiments, experimental testing was conducted. The elements of testing potassium in a buffered solution consist of making reagent, depositing the reagent, preparing the potassium solutions and finally, testing the solutions on the strips. The outlined process below characterizes the basic procedure for how the experimentation was conducted.

First a strip-sensor was produced. The form of this sensor was that of FIG. 2, however many different sensors are possible. Generally, the strip-sensor was prepared according to the following steps: 1) prepare the compound potassium reagent; 2) Using a repeat pipettor, hand deposit 4 μL of reagent onto each sensor (sensors are gold interdigitated electrodes); 4) Dry sensors at 50° C. in convection oven for 5 minutes. In many cases the sensor at this point will be closed up and prepared for testing.

Second, potassium solution was prepared. In order to do so, a buffer solution was made using 50 mM MOPS and 30 mM PEP at pH 7.4. To this buffer stock, a series of target potassium solutions at 2, 3, 4, 5, 6, 7.3, 9, and 10 mM potassium was prepared.

Subsequently potassium testing was conducted. A custom potentiostat test stand was created using test strips according to FIG. 2 and meters/circuitry that interfaced with the strips. When the strip-sensor is produced commercially, a meter will take the place of the petentiostat test stand of course. Each potassium solution across 20 sensors (N=20) was evaluated.

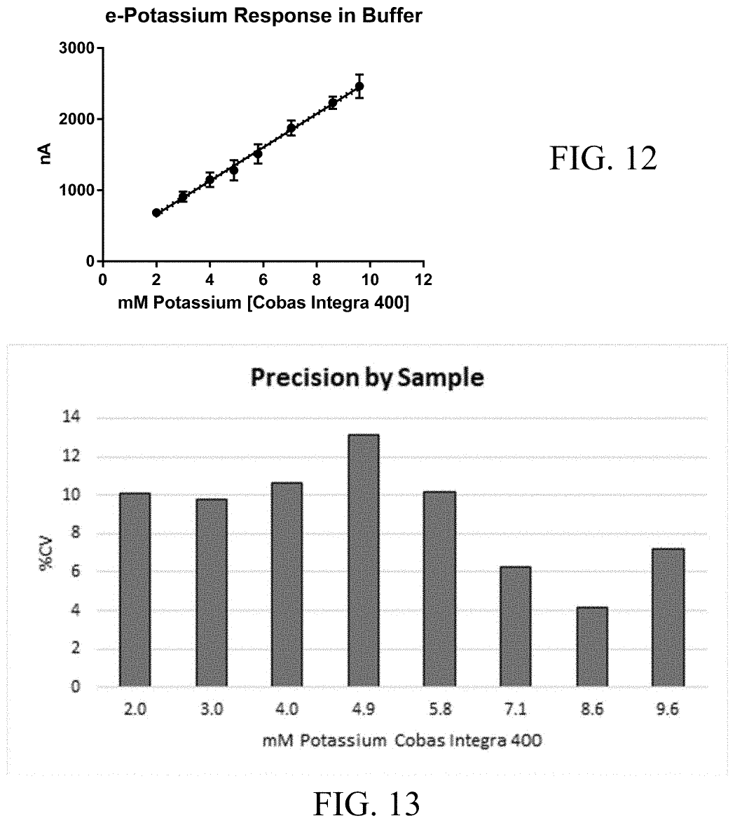

As part of this analysis, linearity and dynamic range was explored. Because potassium is a cofactor of pyruvate kinase, the amount of signal is based on the amount of pyruvate generated over a given amount of time. FIG. 12 shows the results of testing twenty replicates of potassium solutions in a buffered solution. Error bars are representative of standard deviation.

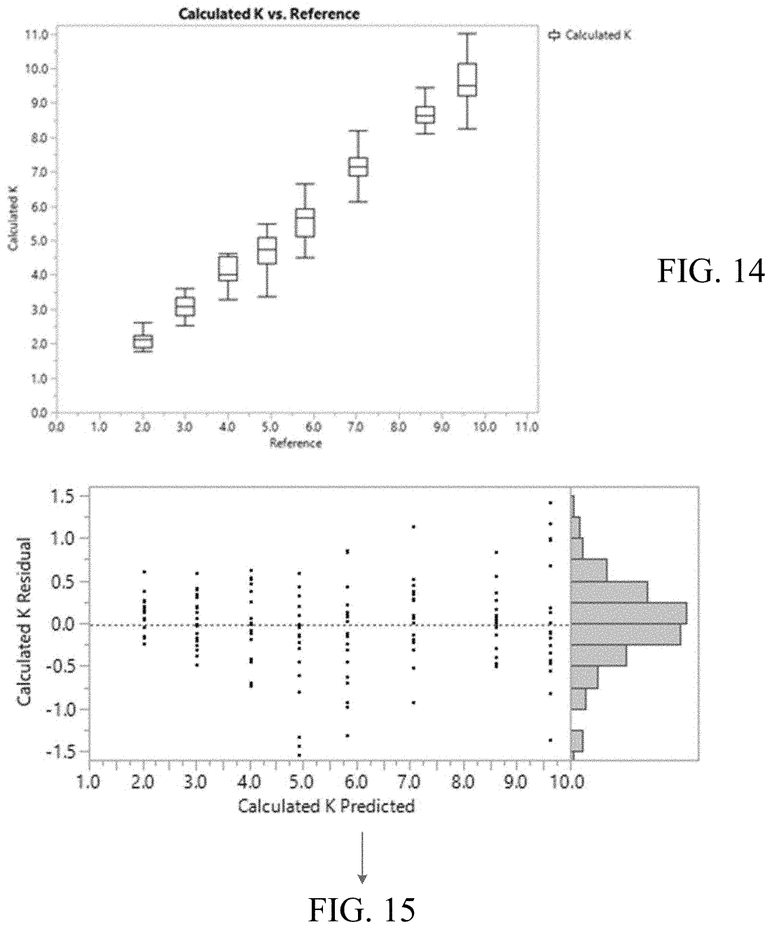

As part of this analysis, the precision was evaluated. By using the equation of the linear regression, the observed potassium values were able to be calculated. From these values the precision was computed for each of the samples. The overall precision was 8.93% CV. Some of the imprecision is due to flyers from hand deposited sensors. FIGS. 13-15 analyze precision in various formats. FIG. 13 shows a graphical representation of the % CV for each of the samples. FIG. 14 shows a box and whisker plot of the potassium detection data. FIG. 15 shows a bias plot of potassium response.

If one were to allow the reaction to go on indefinitely, eventually the same amount of signal would be generated regardless of the potassium concentration. The amount of potassium determines the rate of pyruvate generated. Because of this, the timing of the assay should be determined such that there is sufficient differentiation between the smallest and largest concentration of potassium. Recent testing has shown the best results to be between 60 and 75 seconds. This timing is by no means fixed and is dependent on the setup of the test strip.

The precision reported is good with an average % CV of 8.93%, which will improve significantly, with a commercial system that provides for standardized product of test strips.

Therefore, in many embodiments a test strip from the detection of potassium is provided. This test strip in many embodiments reacts ADP with phosphoenolpyruvate in the presence of potassium ion and pyruvate kinase to produce pyruvate and ATP. Then the produced pyruvate is reacted with phosphate and mediator in the presence of pyruvate oxidase to yield acetylphosphate and reduced mediator. Then the reduced mediator is measured electrochemically and correlated to the amount of reduced mediator to an amount of potassium based on calibration curves.

In many embodiments, a test strip is provided based on this disclosure and interference testing. The test strip includes an electrode area. At the electrode area a reagent mixture is provided. In many embodiments, the reagent mixture includes ADP, Phosphoenolpyruvate, Pyruvate Kinase, Mg2+, Phosphate, a Mediator (nitrosoaniline), and pyruvate Oxidase. In many embodiments, additional agents are added to stabilize and assist in the reaction. In one embodiment, Polyethelyene oxide is also used. This functions as a binder that holds reagent on strip. Examples of some substitutes include Natrasol, Carboxymethyl Cellulose, Xanthum Gum, Polyvinyl Alcohol, Hydroxypropyl Cellulose, Hydroxymethyl Cellulose, etc. Additionally, Triton X-100 (4-(1,1,3,3-Tetramethylbutyl)phenyl-polyethylene glycol, t-Octylphenoxypolyethoxyethanol, Polyethylene glycol tert-octylphenyl ether) at 20% may be included. This provides for reconstitution of the deposited enzymes. Additionally, in many embodiments, lithium phosphate is included in the reagent mixture. This particular addition is included since it can function as both a buffer and a reactant as explained above. In alternatives, less lithium phosphate could be used, and an additional buffer of another type could be included. Lithium phosphate is chosen because it does not include sodium or potassium, which as discussed above, might interfere with the reaction. Additionally, lithium hydroxide is used to adjust the pH of the mixture instead of sodium hydroxide. As previously stated, sodium may interfere with the reaction. In many embodiments, magnesium sulfate is used to provide a source of Mg+2. Additionally, in many embodiments, ADP Na Salt is used. Other sources are possible. An ADP sodium salt is used because some of the other ADP sources have been contaminated with potassium. It will be possible in many embodiments to use a different ADP source. In many embodiments Phosphoenol pyruvate tricyclodexylammonium salt is used as part of the reagent mixture. Other Phosphoenol pyruvates may be used as long as they are not contaminated by potassium. The choice of the mediator may vary. In many embodiments, the mediator may be 4-nitrosoaniline. In other embodiments, it may be a combination of mediators. In some embodiments, potassium ferricyanide is modified via an ion exchange column, to substitute another ion for the potassium. In many cases, this may be with lithium and yield lithium ferricyanide. In some embodiments, it is possible to utilize sodium ions, since sodium largely does not affect the reaction scheme, as noted above. In some embodiments, Rubidium may be exchanged for potassium.

Table 1 below shows one possible reagent mixture. A particular known operative formula is provided as well as useful ranges for the reagents, since depending on the setup of the system and electrodes the concentrations may vary. Below, Q.S. stands for Quantum satis. The Ck pH 6.5 LiOH indicates that LiOH is used to adjust the pH to 6.5. This provides an example of a working system, however, realize that many aspects of the system may be adjusted for purposes or precision, accuracy, and the specifics of the platform it is used on.

TABLE 1
One embodiment of a reagent mixture.

| Reagent | mM | MW | Qty | Units | Useful Ranges | |
|---|---|---|---|---|---|---|
| DI Water | | | 800 | g | | |
| Polyethelyene oxide | | | 10 | g | 0.1-5% | |
| Triton X-100 20% | | | 5 | ml | 0.05-1% | |
| Lithium Phosphate | 50 | 103.93 | 5.1965 | g | 5-200 | mM |
| Ck pH 6.5 | | | | | | |
| use LiOH | | | | | | |
| Magnesium Sulfate | 40 | 246.48 | 9.8592 | g | 5-100 | mM |
| ADP Na Salt | 4 | 427.2 | 1.7088 | g | 2-20 | mM |
| Ck pH 6.5 LiOH | | | | | | |
| Phosphoenol | 30 | 465.58 | 13.9674 | g | 2-100 | mM |
| pyruvate | | | | | | |
| tricyclodexyl- | | | | | | |
| ammonium salt | | | | | | |
| 4-nitrosoaniline | 75 | 150.18 | 11.2635 | g | 10-200 | mM |
| Pyruvate Oxidase | | | 100 | Ku | 50-500 | Ku |
| Pyruvate Kinase | | | 200 | Ku | 100-500 | Ku |
| Q.S. to 1000 ml | | | 1000.0000 | ml | | |
| with DI water | | | | | | |

While specific embodiments have been described in the foregoing detailed description, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof.

What is claimed as new and desired to be protected by Letters Patent of the United States is:

1. A test strip for detecting potassium in a blood sample, comprising:
a working electrode;
a reference electrode;
a testing area, including the working electrode and the reference electrode;
a reagent mixture, the reagent mixture deposited in association with one of the working electrode, the reference electrode, and the testing area, the reagent mixture including Adenosine diphosphate (ADP), Phosphoenolpyruvate, Pyruvate Kinase, Mg2+, Phosphate, a Mediator, and pyruvate Oxidase.

2. The test strip of claim 1, wherein the mediator is nitrosoaniline.

3. The test strip of claim 1, wherein the reagent further includes lithium.

4. The test strip of claim 1, wherein the Pyruvate Kinase is derived from Bacillus stearothermophilus.

5. The test strip of claim 1, wherein the test strip detects potassium according to the equation: 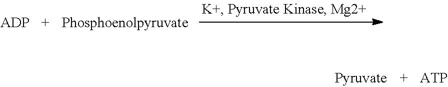 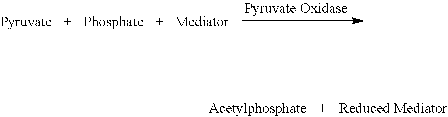
wherein, the reduced mediator represents a charge detectable by a meter.

6. The test strip of claim 1, wherein the mediator is selected from the group consisting of 4-nitrosoaniline, lithium ferricyanide, sodium ferricyanide, and rubidium ferricyanide.

7. The test strip of claim 1, wherein the pH of the reagent mixture is 6.5 and is achieved by adding LiOH.

8. The test strip of claim 1, wherein the reagent mixture further includes polyethelyene oxide and Triton X-100.

9. The test strip of claim 1, wherein the working electrode and the reference electrode are interdigitated.

10. A method for electrochemically detecting potassium in a blood sample comprising:
reacting ADP with phosphoenolpyruvate in the presence of potassium ion and pyruvate kinase to produce pyruvate and ATP;
reacting the produced pyruvate with phosphate and mediator in the presence of pyruvate oxidase to yield acetylphosphate and reduced mediator;
electrochemically measuring the reduced mediator; and
correlating the amount of reduced mediator to an amount of potassium.

11. The method of claim 10 in which the mediator is potassium ferricyanide and the reduced mediator is potassium ferrocyanide.

12. The method of claim 10, wherein the mediator is 4-nitrosoaniline.

13. The method of claim 10, wherein the mediator is lithium ferricyanide.

14. A method of detecting potassium comprising:
reacting ADP with phosphoenolpyruvate in the presence of potassium ion and pyruvate kinase to produce pyruvate and ATP;
reacting the produced pyruvate with phosphate and oxygen in the presence of pyruvate oxidase to yield acetylphosphate and hydrogen in positive ion form, whether bound or unbound;
measuring the hydrogen; and
correlating the amount of hydrogen to an amount of potassium.

15. The method of claim 14, wherein the hydrogen is hydrogen peroxide.

16. The method of claim 14, wherein the hydrogen is bound with a mediator.

17. The method of claim 15, wherein the optically measuring comprises reacting the hydrogen peroxide with a Trinder Reagent.

18. The method of claim 15 wherein the method includes optically measuring and the optically measuring comprises measuring fluorescence.

19. The method of claim 18 wherein the optically measuring comprises reacting the hydrogen peroxide with a Trinder Reagent.

20. The method of claim 13 wherein the reacting steps are performed using a whole blood sample.

21. A test strip for detecting potassium in a blood sample, comprising:
a working electrode;
a reference electrode;
a testing area, including the working electrode and the reference electrode;
a reagent mixture, the reagent mixture deposited in association with one of the working electrode, the reference electrode, and the testing area, the reagent mixture including Adenosine diphosphate (ADP), Phosphoenolpyruvate, Pyruvate Kinase, a Mediator, and pyruvate Oxidase.

22. The test strip of claim 21, wherein the reagent mixture includes Mg2+ and Phosphate.

23. The test strip of claim 21, wherein the mediator is nitrosoaniline.

24. The test strip of claim 21, wherein the reagent further includes lithium.
